(19) United States
(12) Patent Application Publication
(10) Pub. No.: US 2008/0044362 A1
(43) Pub. Date: Feb. 21, 2008

(54) COSMETIC COMPOSITION COMPRISING BLOCK COPOLYMER TEMPLATED METAL OXIDE PARTICLES

(75) Inventor: Murat Quadir, Scotch Plains, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/759,619

(22) Filed: Jun. 7, 2007

Related U.S. Application Data
(60) Provisional application No. 60/817,711, filed on Jun. 30, 2006.

(51) Int. Cl. A61K 8/04; A61K 8/19; A61K 8/27; A61Q 99/00

(52) U.S. Cl. 424/47; 424/59; 424/600; 424/642

(57) ABSTRACT

Disclosed are cosmetic compositions containing a metal, a polymer having a non-functional domain and a functional domain having a plurality of functional groups that interact with the metal, and a cosmetically acceptable carrier. Also disclosed are methods and systems for applying the composition to keratinous substrates, whereby aggregates of the metal are formed or deposited on the substrate.

### STATEMENT OF RELATED APPLICATIONS

This application is a non-provisional application of, and claims benefit to, U.S. Provisional Application No. 60/817,711, filed Jun. 30, 2006.

### BACKGROUND OF THE INVENTION

EP-A-1 082 952 discloses make-up having a sparkling and metallic appearance containing glass particles covered with a metal layer.

EP-A-953 330 discloses cosmetic compositions exhibiting iridescent effects containing a goniochromatic colorant having multilayer metal particles able to produce different colors and a monochromatic colorant having one of the colors of the goniochromatic colorant.

WO-A-02/03913 discloses nail enamel compositions having a mirror-like appearance containing aluminum particles in the form of platelets and high molecular weight film-forming agents.

US 2005/0112154 discloses a cosmetic composition containing particles having a metal core and an agent exhibiting cosmetic activity for contributing sheen to keratinous substances.

### SUMMARY OF THE INVENTION

A first aspect of the present invention is directed to a cosmetic composition for application to a keratinous substrate, comprising a polymer, a metal, and a cosmetically acceptable carrier. The polymer contains a non-functional domain and a functional domain that contains functional groups that interact with the metal, which results in the formation of metallic aggregates.

A second aspect of the present invention is directed to a method of applying the cosmetic composition to a keratinous substrate. In some embodiments, the method entails an additional step of applying to the keratinous substrate a developer containing an oxidizing agent, which after evaporation or removal of the carrier converts the metal to another form.

A further related aspect of the present invention is directed to a system for treating a keratinous substrate. The system includes a first package comprising a composition containing a polymer, a metal, and a cosmetically acceptable carrier, and a second package containing an oxidizing agent.

The inventive compositions and systems provide one or more cosmetically aesthetic optical properties when applied to keratinous substrates. Such properties include enhanced luster, gloss, coloration, reflectance, glowing effects and an enhancement of natural color.

### DETAILED DESCRIPTION

The present invention is directed to a cosmetic composition for application to a keratinous substrate, e.g., natural or synthetic hair or skin. In some embodiments, the composition is applied to hair in the form of a mousse or gel. Interactions between the polymer and the metal result in the deposition or formation of aggregates of the metal on the substrate upon or after application of the composition.

Polymers useful in the present invention contain at least two domains—a functional domain and a non-functional domain. In some embodiments, the functional and non-functional domains are comprised of different monomers, in which case the polymer of the invention is considered a block copolymer. A “block copolymer” is a polymer comprising molecules in which there is a linear, branched, or networked arrangement of blocks, wherein each “block” contains monomers at least some of which have at least one structural feature absent from the adjacent portions.

The functional domain contains a plurality of functional groups that interact with a metal. The interaction results in deposition or formation of metallic aggregates (clusters, deposits, or concentrations of interacting polymer and metal) on the substrate. The number and type of the specific monomers comprising the functional domain may vary. In some embodiments, the function domain contains a plurality of the same monomer, and thus may be considered a homopolymer in and of itself. In yet other embodiments, the function domain is copolymeric in nature, and contains two or more different monomers, each having the same or different functional groups. In some embodiments, each monomer in the functional domain contains a functional group. In other embodiments, however, one or more monomers in the functional domain do not have a functional group. In some embodiments, the functional domain is relatively polar in nature compared to the non-functional domain.

Functional groups useful in the present invention include hydroxyl, amide, amine, azo, carbonyl, carboxy, ester, nitro, nitroso, thiol, and imine groups. In some embodiments, the functional domain is comprised of monomers having at least one carboxyl functional group. Examples of such monomers include norbornene carboxylic acid and norbornene dicarboxylic acid.

The functional groups that interact with the metal are not limited and can be any compound that is capable of forming at least one bond with a metal. Non-limiting types of compounds include catechol, a substituted catechol, electron-donating bidentate ligands, dihydroxy cyclobutenedione, ascorbic acid, methyl catechol, tertbutyl catechol, salicylic acid, a substituted salicylic acid, gallol, a substituted gallol, benzene-1,2,3-triol, a substituted benzene-1,2,3-triol, benzene-1,2,4-triol, a substituted benzene-1,2,4-triol, benzene-1,2,3,4-tetraol, a substituted benzene-1,2,3,4-tetraol, dopamine, alizarin, an organoamine, an organopolycarboxylate, an organoamino carboxylate, an organosilane, an alkoxysilane, an organophosphate, an organophosphonate, an aminophosphonate, a disulfide, a mercaptocarboxylate, an alkylthiocarbamate, an alkylcarbamate, a polyalkylene glycol/amino acid copolymer, a mPEG-DOPA copolymer, poly[(3,4-dihydroxystyrene)/styrene copolymer, a polyalkylene glycol functionalized acrylic acid, a polyalkylene glycol functionalized methacrylic acid, a gallol-PEG polymer, a compound or polymer with adjacent hydroxyl groups and mixtures thereof. Other useful polymers include both natural and synthetic polymers containing at least one catechol group. Examples of these types of polymers are exemplified in the publications listed below.

The substituient on the substituted groups listed above is not limited. Examples of substituients on the catechol and salicylic acid include the following groups amino, alkyl amino, N-substituted amino, —O-alkyl, —O-aryl, alkyl, alkenyl, alkynyl, cycloalkyl, heterocycloalkyl, aryl, heteroaryl, alkylaryl, alkyl-substituted cycloalkyl, alkyl-substituted heterocycloalkyl, aralkyl, alkylheteroaryl, heteroarylalkyl. The substituients listed above may also be oligomers and polymers. The substituient oligomers and polymers may be dopamine based such as mPEG-DOPA oligomers and polymers. The substituent oligomers and polymers may be a polyalkyl glycol such as PEG.

Additional useful substituents and substituted catachol compounds may be found in Sheng, et al., J. Am. Chem. Soc., 2006, 128, 10676-10677; T. Rajh, et al., J. Phys. Chem. B 2002, 106, 10543-10552; J. Dalsin, et al., Langmuir 2005, 21, 640-646; C. Xu, et al., J. Am. Chem. Soc. 2004, 126, 9938-9939; X. Fan, et al., Composite Science and Technology 66 (2006) 1195-1201; J. Dalsin, et al., Materialstoday, September 2005, 38-46; H. Lee, et al., PNAS, Aug. 29, 2006, Vol. 103, No. 35, 12999-13003; G. Westwood, et al., Macromolecules, Published on Web May 4, 2007; B. P. Lee, et al., Macromolecules, 2006, 39, 1740-1748; P. Podsiadlo, et al., Advanced Materials, 19, 7, Apr. 2007, 949-955; H. Gu, J. Am. Chem. Soc., 2005, 127, 34-35. The contents of the above publications are herein incorporated by reference.

Like the functional domain, the non-functional domain may be comprised of the same or different monomers. It is structurally different than the functional domain, however, in that it does not form metallic aggregates, or at least to the same degree as the functional domain. In some embodiments, the non-functional domain contains one or more different monomers that do not contain functional groups. In other embodiments, the non-functional domain contains monomers that are non-polar or that are less polar than the monomers comprising the functional domain such that metals selectively or preferentially bind to the functional domain. In some embodiments, the non-functional domain contains monomers of norbornene.

The size of the non-functional domain in relation to the size of the functional domain is not critical. In some embodiments, where the number of monomers contained in the non-functional domain is greater than the number of monomers contained in the functional domain, such that there is a greater proportion of non-functional domain, the metallic aggregates tend to be distributed and dispersed within a matrix of a non-functional domain polymer. Similarly, a relatively large functional domain tends to result in an increase in the size and/or enhance the optical properties of the metallic aggregates that are formed.

The spatial arrangement of the functional domain in relation to the non-functional domain may also vary. The functional domain may be attached to the non-functional domain in a linear manner such that the polymer contains alternating functional domains and non-functional domains. Alternatively, two or more functional domains may be linked to a common central non-functional domain just as a single functional domain may be flanked by multiple non-functional domains.

The ratio of the number of monomers comprising the functional domain to the number of monomers comprising the non-functional domain generally ranges from about 20:1 to about 1:20. In some embodiments, the ratio ranges from about 10:1 to about 1:10. In further embodiments, the ratio ranges from about 8:1 to about 1:8, and yet in other embodiments, the ratio ranges from about 3:1 to about 1:3.

The polydispersity indexes of the polymers of the present invention generally range from about 1 to about 2, and in some embodiments, from about 1 to about 1.5, or from about 1 to about 1.2. Polydispersity indexes (“PDI”) are a measure of the distribution of molecular weights in a given polymer sample. The PDI is calculated as the weight average molecular weight divided by the number average molecular weight.

The amount of polymer present in the cosmetic compositions of the current invention generally ranges from about 0.05% to about 30%, and in some embodiments, preferably from about 1% to about 10%, based on the total weight of the composition.

The metal present in the composition may be in the form of a metal salt, a metal cation, or a metal oxide. Metal salts according to the present invention result from the ionic bonding of a metal cation with an anion. Moreover, the metal can combine directly with a nonmetal to form a metal salt. The metal salts of the present invention may be inorganic or organic. In a preferred embodiment of the present invention, the metal salts are inorganic metal salts. In some embodiments of the present invention, the metal cation constituting the metal salt is selected from Groups IIA, IIB, VA, and IIIB through VIIIB of the periodic table of elements, regardless of oxidation state. In some embodiments, the metal cation is selected from Be, Mg, Ca, Sr, Zn, Cd, Hg, N, P, Sb, Mn, Fe, Co, Ni, Cu, Mo, Pd, Ag, Au, Zr, and Ti, regardless of oxidation state. In some other embodiments, the metal cation is selected from Zn, Fe, and Ca, regardless of oxidation state.

The anion of the metal salt may be selected from the groups including Cl⁻, SO₄²⁻, SO₃²⁻, PO₄₋, HPO₄²⁻, CrO₄²⁻, CR₂O₇²⁻, C₂O₄²⁻, S₂O₃²⁻, NO₃⁻, NO₂⁻, CIO₄⁻, CIO₃⁻, CIO₂⁻, OCl⁻¹, CH₃COO⁻, IO₃⁻ and I₂⁻. In some embodiments, the anion is chosen from Cl₂, SO₄²⁻, SO₃²⁻, PO₄³⁻, and HPO₄²⁻.

In general, the amount of metal in the composition varies in relation to the amount of polymer. Thus, a metal-to-polymer ratio generally ranges from about 30:1 to about 1:30, and in some embodiments, from about 20:1 to about 1:20, and yet in others from about 10:1 to about 1:10.

The manner in which the metals of the present invention interact with the functional domains of the polymer is not critical. The term “interact”, as used herein, may refer to ionic bonding, covalent bonding, metallic bonding, coordinate covalent bonding, complexation, hydrogen bonding, van der waals interactions, coordination, chelation, association, or physical entrapment. Regardless of the exact mechanism of the interaction, the metal selectively interacts with functional groups in the functional domains, resulting in aggregates of functional domain enriched with metal that is deposited on or is formed after composition is applied to the substrate.

Interactions between the functional groups of the functional domain and metals may occur intra-molecularly or inter-molecularly, such that interactions occur, for example, between functional groups within the same functional domain on the same polymer strand, between other functional domains on the same polymer strand, or even between function domains on different polymer strands.

Once formed, the metallic aggregates may have different shapes. For example, the metallic aggregates may take the form of micelles, lamellar structures, sheets, spheres, flakes, fibers, tubes or cylinders. On the other hand, the metallic aggregates may have entirely random shapes. Indeed, each of the metallic aggregates may have different shapes and spacing on the keratinous substrate.

The size of the metallic aggregates can be varied depending on the chemical structure of the polymer, the average molecular weight of the polymer, the monomer ratio and content of the polymer, the polydispersity index of the polymer, the reactive functionality of the units of the polymer, and the cosmetically acceptable carrier as well as the metal itself. As the sizes of the metallic aggregates change, various colors or hues may be realized when such metallic aggregates are deposited on a keratinous substrate. Thus, a variety of colors, highlights, or tints may be achieved simply by varying the size of the metallic aggregates.

The polymers of the present invention can be synthesized in accordance with standard techniques. Next, the polymer is mixed with a cosmetically acceptable carrier. An appropriate amount of metal is then added to the polymer solution such that the metal may interact with the functional groups of the functional domain. After the carrier is at least partially evaporated and/or after the solution is applied to a keratinous substrate, metallic aggregates dispersed in a polymeric film will form.

The compositions of the present invention also include at least one cosmetically acceptable carrier. A cosmetically acceptable carrier refers to any organic or aqueous solvent or solvent system that is compatible with the other components of the invention and suitable for human use. However, persons skilled in the art would appreciate that not every carrier for the polymer and metal is “cosmetically acceptable”. Examples of non-cosmetically acceptable carriers include tetrahydrofuran, dimethyl sulfoxide, benezene, benezene derivatives, and dimethylformamide. In addition, carriers that are toxic, abrasive, or in any way damaging to keratinous substrates should not be utilized.

Generally, cosmetically acceptable carriers may be selected from volatile organic solvents, non-volatile organic solvents, water, and mixtures thereof. Alcohols that may be utilized as carriers include C1 to C20 straight chain, branched, or cyclic mono-alcohols, including ethanol, propanol, butanol, tert-butanol, isopropanol and mixtures thereof, polyalcohols (e.g., diethylene glycol), glycol ethers and alkyl ethers of glycol or of diethylene glycol. Other cosmetically acceptable carriers such as triglycerides (e.g., glycerin), fatty alcohols derived from natural fats and/or oils (e.g., 1-dodecanol and 1-octadecanol), hydrocarbons (e.g., mineral oils, mineral solvents, mineral spirits, petroleum, waxes, synthetic hydrocarbons, animal oils, vegetable oils, and volatile hydrocarbons (e.g., isododecane)), light paraffinic solvents, and non-hydrocarbon solvents (e.g., amyl acetate, butyl acetate, isobutyl acetate, ethyl acetate, propyl acetate and isopropyl acetate) may also be useful.

In preferred embodiments, the cosmetically acceptable carrier is selected from an alcohol or water or a mixture thereof.

The amount of cosmetically acceptable carrier generally ranges from about 1% to about 99% by weight and in some embodiments from about 2% to about 80% by weight, based on the total weight of the composition.

The cosmetic compositions of the present invention may be formulated in a variety of products, such as lip stick, lip pencil, blush, foundation, rouge, skin creams, nail polish, nail conditioner, hair dye product, mascara, spray, mousse, styling gel, leave-on conditioner, shampoo, conditioner, cream rise mascara, permanent waving composition, hair care product, hair spray, hair dye product, hair treatment product, and hair styling product.

In some embodiments, the personal care product of the current invention is a hair styling product, particularly a styling gel or mousse. Accordingly, in these embodiments, the compositions typically contain one or more propellants, gelling/thickening agents, surfactants, film forming agents, silicone compounds, conditioning agents and lubricating agents.

Suitable propellants include volatile hydrocarbons, such as n-butane, propane, isobutane and pentane, chloro and/or fluoro hydrocarbons and mixtures thereof. Carbon dioxide, nitrous oxide, dimethyl ether, nitrogen or compressed air may also be used.

Gelling agents include natural or synthetic carboxyvinyl polymers (carbomer) such as polyvinylpyrrolidone (PVP) and polyvinyl alcohol, clays and modified clays (montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite), acrylic copolymers (e.g., crosslinked acrylates (e.g. Carbopol 982)), and hydrophobically-modified acrylates (e.g. Carbopol 1382); polyacrylamides, carbonates such as propylene carbonate, cellulose derivatives, natural gums, polysaccharides and resins thereof, silicas, silicates, silica silylate, and derivatives thereof.

Surfactants useful in the present invention include non-ionic and amphoteric surfactants or mixtures thereof. In the case of mousse, surfactants may act as a foaming agent. The amphoteric surfactants useful in the present invention include betaines, sultaines, hydroxysultaines, alkyl amphodiacetates, alkyl amphodipropionates, and imidazolines, or salts thereof. Other fatty acid condensates such as those formed with amino acids, proteins, and the like are suitable. Amphoteric surfactants are typically available for commercial sale in solution form with the active surfactant accounting for approximately 40% of the total solution weight. Cocamphodipropionate is particularly preferred, for example, MIRANOL C2M-SF Conc. (disodium cocamphodipropionate), in its salt-free form, available from Rhone-Poulenc. MIRANOL is sold in solution form with amphoteric surfactants containing approximately 40% of the total solution weight; for example, 10 g of MIRANOL contain about 4 g of amphoteric surfactant. CROSULTAINE C-50 (cocamidopropyl hydroxysultaine) is available from Croda. CROSULTAINE is also sold in solution form with the amphoteric surfactant composing approximately 50% of the total solution weight. Other amphoteric surfactants that may be useful in the present invention include disodium wheatgermimido PEG-2 sulfosuccinate, available under the trade name MACKANATE WGD from McIntyre Group Ltd., which is a solution with amphoteric surfactants composing approximately 39% of the total solution weight, and disodium soyamphodiacetate, available under the trade name MACKAM 2S from McIntyre Group Ltd., which is a solution with amphoteric surfactants composing approximately 34.5% of the total solution weight.

Nonionic surfactants useful in the present invention are preferably formed from a fatty alcohol, a fatty acid, or a glyceride with a C8 to C24 carbon chain, preferably a C12 to C18 carbon chain, more preferably a C16 to C18 carbon chain, derivatized to yield a Hydrophilic-Lipophilic Balance (HLB) of at least 10. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 10.

Film forming agents (e.g., film forming polymers) may be added, provided that they are compatible with the polymer containing the functional and non-functional domain(s). Agents that may be useful in the present invention include keratin derivatives, such as keratin hydrolysates and sulphonic keratins; anionic, cationic, amphoteric or nonionic derivatives of chitin or chitosan; cellulose derivatives such as hydroxyethylcellulose, hydropropylcellulose, methylcellulose, ethylhydroxyethylcellulose, carboxymethylcellulose, and quaternized derivatives of cellulose; acrylic polymers or copolymers, such as polyacrylates or polymethacrylates; polyvinylpyrrolidones (PVP) and vinyl copolymers, such as methyl vinyl ether-maleic anhydride copolymers, or vinyl acetate-crotonic acid copolymer; water-dispersible anionic polyesteramide and/or polyester polymers. Polyurethane polymers, especially anionic, cationic, nonionic or amphoteric polyurethanes, acrylic polyurethanes, polyvinylpyrrolidone polyurethanes, polyester polyurethanes, polyether polyurethanes, polyureas, polyurea/polyurethanes, and mixtures thereof; and polymers of natural origin, modified if desired, such as gum arabic, guar gum, xanthan derivatives, karaya gum; alginates and carragheenates; glycoaminoglycans, hyaluronic acid and its derivatives; shellac, sandarac gum, dammars, elemis and copals, may also be useful.

Suitable silicone compounds, which may be included to enhance shine, include silicone oils and elastomers, for example, DC 9011 silicone elastomer blend (cyclopentasiloxane (and) PEG-12 (and) dimethicone crosspolymer), DC 9040 silicone elastomer blend (Cyclopentasiloxane and Dimethicone Crosspolymer), and DC 9506 (Dimethicone/Vinyl Dimethicone Crosspolymer) (all commercially available from Dow Corning).

Suitable conditioning agents include cationic polymers, such as polyquaternium 4, polyquaternium 6, polyquaternium 7, polyquaternium 10, polyquaternium 11, polyquaternium 16, polyquaternium 22, polyquaternium 32, and polyquaternium 52, and other cationic conditioners, such as quaternium 27, behenamidopropyl PG-dimonium chloride, hydroxyethyl tallowedimonium chloride, hexadimethrine chloride, stearalkonium chloride, behentrimonium chloride, and cetrimonium chloride.

Examples of lubicants (lubricating agents) include glycols and polyhydric alcohols such as glycerin, sorbitol, and propylene glycol.

Additionally, compositions of the present invention may contain cosmetically acceptable additives conventionally used in the field of cosmetics including preservatives, anti-oxidants, re-fatting agents, vitamins, chelating agents, fillers, other coloring agents, buffers, fragrances, extracts (e.g., plant extracts), and UV filters/sunscreens (e.g., benzophenone-4). They will also be present in amounts appropriate to the type of personal care product.

The metal aggregates may be formed or deposited in the keratinous substrate simply by applying the composition. In these embodiments, the carrier is a volatile organic solvent. The aesthetic appeal of the aggregates may be enhanced by removing at least some of the cosmetically acceptable carrier, (e.g., especially when the carrier is a non-volatile organic solvent or water. Removal of the carrier may be facilitated by application of heat (e.g., at temperatures ranging from about 20° C. to about 220° C., preferably ranging from about 100° C. to about 200° C., most preferably ranging from about 140° C. to about 190° C.). Heat may be applied through the use of a hair dryer, curling iron, or straightening iron. In other embodiments, removal of the carrier is achieved by application or a second composition containing an additional carrier. In some embodiments, the cosmetically acceptable carrier is washed off the keratinous substrate such that a film is left behind.

In some embodiments, the method entails an additional step of applying to the keratinous substrate a developer composition containing an oxidizing agent. Application of the developer facilitates the conversion of the metal of the metallic aggregate to the respective metal oxide. Oxidizing agents useful in the present invention include weak inorganic bases, such as ammonium hydroxide, calcium hydroxide, barium hydroxide, ammonium hydroxide and organic bases such as weak organic bases (e.g., amino methyl propanol (AMP)).

In yet other embodiments, the metal is converted to the respective metal oxide via the combined use of heat and an oxidizing agent (e.g., a weak or organic base).

The developer and/or heat may be applied to the keratinous substrate before or after application of the cosmetic composition. However, they may also be applied together with the cosmetic composition such that the conversion process begins immediately. In preferred embodiments, the developer and/or heat is applied after application of the cosmetic composition. Regardless of the order in which the developer and/or heat are applied, conversion to the metal oxide may affect luster, gloss, or coloration effects.

The present invention also provides a system for the deposition of metallic aggregates onto a keratinous substrate. Such a system comprises at least two components, each separately packaged. The first component or composition contains a polymer, a metal, and a cosmetically acceptable carrier. The second component, contains an oxidizing agent. The system may further include other products and instructions describing the use of the system.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### EXAMPLES

The following example is intended to illustrative of the invention, but not limiting thereof.

Hair Composition

| Ingredients | Composition | |
|---|---|---|
| Poly(norbornene)₄₀₀-block- | 1.0 | g |
| poly(norbornene dicarboxylic acid)₅₀ | | |
| ZnCl₂ | 0.10 | g |
| Acetone | q.s. to 100 | g |

The diblock copolymer norbornene-block-norbornene dicarboxylic acid (1.0 g) is dissolved in acetone to make a 1.0% (w/v) solution. Approximately 0.10 g of ZnCl₂ is added to this polymer solution. The solution is stirred to allow the Zn⁺² cations to associate with the carboxylic groups on the second block of the copolymer. The final solution is then applied to a hair tress (5 g of the solution to 1 g of hair) at ambient temperature and the hair tress is blow-dried.

1. A cosmetic composition, comprising:
a) a metal,
b) a polymer having a non-functional domain and a functional domain having a plurality of functional groups that interact with said metal, and
c) a cosmetically acceptable carrier.

2. The cosmetic composition of claim 1, wherein said metal is in the form of a metal salt, a metal oxide, or a metal cation.

3. The cosmetic composition of claim 1, wherein said metal is zinc.

4. The cosmetic composition of claim 1, wherein said functional groups comprise carboxylic acid groups.

5. The cosmetic composition of claim 1, wherein said functional domain comprises repeating units of norbornene carboxylic acid.

6. The cosmetic composition of claim 1, wherein said functional domain comprises repeating units of norbornene dicarboxylic acid.

7. The cosmetic composition of claim 1, wherein said non-functional domain comprises repeating units of norbornene.

8. The cosmetic composition of claim 1, wherein said functional domain comprises repeating units chosen from catechol, substituted catechol, and mixtures thereof.

9. The cosmetic composition of claim 1, wherein said functional domain comprises repeating units of a substituted salicylic acid.

10. The cosmetic composition of claim 1, wherein said carrier comprises a volatile organic solvent.

11. The cosmetic composition of claim 1, further comprising a propellant.

12. The cosmetic composition of claim 1, further comprising a thickener or gelling agent.

13. The cosmetic composition of claim 9 or 10, further comprising one or more cosmetically acceptable ingredients selected from the group consisting of silicone oils and elastomers, conditioning agents, amphoteric or non-ionic surfactants, lubricants and sunscreen agents or UV filters.

14. A method of treating a keratinous substrate comprising:
a. applying onto the substrate a cosmetic composition comprising:
a metal,
a polymer having a non-functional domain and a functional domain having a plurality of functional groups that interact with the metal, and
a cosmetically acceptable carrier,
b. wherein the applying results in formation or deposition of aggregates of the metal on the keratinous substrate.

15. The method of claim 14, wherein the carrier comprises a volatile organic solvent, and the aggregates form upon evaporation of the carrier.

16. The method of claim 14, wherein the carrier comprises a non-volatile solvent, and the aggregates form upon removal of the carrier.

17. The method of claim 16, wherein the removal of the carrier is carried out by heating.

18. The method of claim 14, further comprising the step of applying to the keratinous substrate a composition comprising an oxidizing agent.

19. The method of claim 18, wherein the oxidizing agent comprises a weak base.

20. The method of claim 18, wherein said oxidizing agent is applied to the substrate after applying the cosmetic composition.

21. A system for treating a keratinous substrate, comprising a first container comprising a metal, a polymer having a non-functional domain and a functional domain having a plurality of functional groups that interact with the metal, and a cosmetically acceptable carrier, and a second container comprising a developer composition comprising an oxidizing agent.
